(19) United States
(12) Patent Application Publication
(10) Pub. No.: US 2022/0280570 A1
(43) Pub. Date: Sep. 8, 2022

(54) STROMAL CELLS DERIVED FROM NEONATAL FELINES AND USES THEREOF

(71) Applicant: VETBIOBANK, Marcy L'Etoile (FR)

(72) Inventors: Stéphane MADDENS, Roquefort La Bedoule (FR); Nathalie SAULNIER, Ecully (FR); Marine FEBRRE, L'Arbresle (FR)

(21) Appl. No.: 17/422,010

(22) PCT Filed: Jan. 13, 2020
(86) PCT No.: PCT/EP2020/050721

§ 371 Date: Jul. 9, 2021

(30) Foreign Application Priority Data
Jan. 11, 2019 (FR) 19 00284

(51) Int. Cl. A61K 35/28; A61P 1/02; A61K 47/69; A61K 9/00

(52) U.S. Cl. CPC A61K 35/28; A61P 1/02; A61K 47/6903; A61K 9/0019

(57) ABSTRACT

The present invention relates to a pharmaceutical composition comprising a population of stromal cells derived from neonatal felines and a pharmaceutically acceptable vehicle, in particular a solution comprising a cryoprotectant. The invention also relates to a population of neonatal stromal cells or a pharmaceutical composition comprising such a population for use in cell therapy, in particular allogeneic cell therapy, and more particularly for treating feline stomatitis. The invention also relates to a ready-to-use injectable solution comprising stromal cells derived from neonatal felines.

### TECHNICAL FIELD

The invention relates to a population of feline neonatal stromal cells, a pharmaceutical composition and an injectable solution comprising said cells, as well as a procedure for obtaining this composition from neonatal tissue. The invention also relates to the use of these cells and compositions in cell therapy in felines, in particular for the treatment of stomatitis.

### Prior Art

Oro-dental diseases are common in cats and are in general associated with halitosis, dysphagia and dysorexia that can lead to dehydration and emaciation of the animal. Moreover, there are inflammatory manifestations (ulcerative, ulceroproliferative) of the oral mucosa, particularly in the caudal region, becoming chronic and resistant to conventional treatments. These phenomena are routinely designated feline chronic gingivostomatitis (CGS), or chronic stomatitis, lympho-plasmocytic stomatitis or granulomatous stomatitis. The prevalence of CGS is approximately 0.7 to 12% of the feline population according to the study (1; 2). CGS manifests itself by several symptoms such as oral ulcers, inflammatory lesions, hypersalivation, weight loss in the animal or bleeding. This debilitating disease is responsible for pain, refusal to eat, infectious complications and is the top cause of dental avulsion in cats. This operation is serious and costly and requires complementary therapy in 69% of cases. Moreover, among the subjects having undergone this avulsion, 32.6% of cats remain resistant to treatment (3) (Jennings et al., 2015). The etiology of CGS is still poorly understood, but it appears to have multiple factors and it underlies mainly a local dysimmunitary component (4). The strong presence of T cells at the lesion sites suggests an exacerbation of the immune reaction of the host with respect to the mucosa. The treatments such as the use of antibiotics, of corticoids, of interferons, of anti-inflammatories or of non-steroidal anti-inflammatories (NSAIDs) do not show a reproducible effectiveness against CGS and make it a disease with a progression that cannot be controlled. Stromal cells represent a promising alternative in this pathological context. The latter have an immunomodulation capacity and are capable of regulating the local and/or systemic immune activity by limiting the proliferation and the differentiation of the cells responsible for the inflammation (lymphocytes, T, B, polynuclear, monocytes, dendritic cells) (5). In the context of CGS, feline stromal cells have already been used by a person skilled in the art as a cell therapy (6; WO2017/062475A1). In these documents, a treatment consisting of a double administering of 20·10⁶ feline stromal cells coming from adipose tissue at an interval of one month demonstrated an effectiveness of approximately 70% in the context of autologous therapies and of approximately 57% in the context of allogenic therapies (7; US20160199414A1), on pathological subjects not responding to conventional therapies. The cells used in these documents are fresh cells, that is to say that have not undergone a step of cryopreservation or which are re-cultured after cryopreservation in the case of the second injection of the subject. Moreover, in these documents, the collection of the feline stromal cells requires the removal of adipose tissue in an individual and thus a surgical operation is necessary, limiting the industrialisable nature of a therapy using the properties of these cells. During the observation of symptoms of feline CGS, very often all of the teeth of the animal are extracted. In certain cases, a partial dental avulsion can be carried out. For example, when the oral lesions are not generalised, the canines can be preserved. Likewise, when the inflammation is localised, it is possible for the dental extraction to only relate to the teeth affected by the lesions.

At present, the treatment of feline CGS by administering feline stromal cells is systematically preceded by a total dental avulsion.

### Objectives

One objective of the invention is to provide a product for the treatment of feline stomatitis that is easy to use, available at any time and industrialisable.

Another objective of the invention is to provide a population of cells intended for cell therapy, in particular for allogenic therapeutic uses, and more particularly for the treatment of tissular injuries of the mucosa in felines such as feline stomatitis.

Another objective of the invention is to provide a population of cells having a good capacity for proliferation to be able to be produced on the industrial scale and which preserves all of these biological properties after a step of cryopreservation.

Another objective is to provide a method for obtaining a ready-to-use pharmaceutical composition comprising said cell population.

Another objective is to provide a pharmaceutical composition comprising said cell population.

Another objective of the invention is to develop a ready-to-use injectable solution which is directly usable for a therapeutic use, without needing to re-culture the cells or change the cell medium before injection into the subject.

The inventors have discovered in a surprising manner that feline neonatal stromal cells allowed to treat oral inflammations of the mucosae in felines in an effective and durable manner, in an allogenic context, in particular by the injection of a single dose to the subject to be treated.

Moreover, they discovered that this treatment was effective in subjects having undergone a total dental avulsion or not, or having undergone a partial dental avulsion.

Indeed, this cell population is capable of exerting a localised and/or overall anti-inflammatory action allowing to substantially reduce the oral lesions. This anti-inflammatory activity is related, inter alia, to the immunomodulating properties of the feline neonatal stromal cells. These properties can be defined as the capacity of the feline neonatal stromal cells to inhibit the differentiation, the proliferation and/or the activity of the immune cells responsible for the inflammatory context of a tissue or when these immune cells are cultivated in vitro.

These cells also have a strong proliferation potential thus allowing their multiplication in vitro on the industrial scale. Moreover, the removal of this type of cell does not necessarily require an invasive step.

### Disclosure of the Invention

### Cell Population

A first object of the present disclosure relates to a population of feline neonatal stromal cells (NSCs). In the sense of the present invention, “feline” means any animal of the family Felidae, in particular any animal of the genus Felis, and more particularly the cat, Felis silvestris catus.

In a preferred embodiment, the feline according to the invention is a cat.

### Origin of the Feline NSCs

“Neonatal stromal cells” means any cell having one, several or all of the characteristics of the mesenchymal stem cells and having a neonatal origin. The term “neonatal” means that the feline NSCs can be isolated from all of the tissular and/or biological fluid sources, which come from the extra-embryonic membranes and can be recovered during the phase of parturition/Caesarean section or during the gestation period.

In a specific embodiment, these neonatal tissular sources coming from the extra-embryonic membranes also called foetal membranes correspond to the umbilical cord and more particularly to the matrix of the umbilical cord such as Wharton's jelly, the perivascular zone of the arteries and/or the umbilical vein. In another specific embodiment, the neonatal tissue corresponds to the amniotic membrane or more particularly to the epithelium of the amniotic membrane and/or of the amnion. In another specific embodiment, the neonatal tissue corresponds to the placenta which is of the endotheliochorial type or more particularly to the chorionic plate/chorionic mesoderm, to the chorionic trophoblast/trophoblast, to the chorionic villi, to the placental cotyledons, to the placental decidua and/or to the perivascular system of the placenta.

This neonatal tissue is considered to be surgical waste during births and thus does not involve an additional surgical operation to recover it.

Therefore, it can be recovered aseptically during Caesarean sections or post-partum during a natural birth in gestating females. For example, as soon as the newborn has exited the amniotic sac and is safe, the extra-embryonic tissue is immediately transferred into a transport box containing for example a saline solution buffered with Dulbecco's phosphate to be transported to the laboratory.

In an alternative embodiment, the tissue of extra-embryonic membranes can be recovered throughout the gestation period of the female cat (on average 64-69 days long) in the context of an elective ovariohysterectomy which is a routine operation aimed at abortion and sterilisation.

“Biological fluids” means fluids of extra-embryonic membranes such as the blood of the umbilical cord or the amniotic fluid and/or placental blood. The blood of the umbilical cord can be recovered by draining the umbilical cord. As for the amniotic fluid, it can be recovered by draining the liquid contained in the amniotic sac or by piercing the amniotic sac and by pouring the amniotic fluid that it contains into a tube, a Petri dish or any other suitable container.

According to a specific embodiment of the invention, said population of feline NSCs comes from a sample of neonatal tissue, in particular from one or more placentas and/or from one or more umbilical cords; or from a sample of neonatal fluid, in particular of blood of one or more umbilical cords or of amniotic fluids.

The feline NSCs coming from this tissue and fluids has the advantage of being only slightly exposed to exogenic stresses that can induce epigenetic modifications. In a specific embodiment, the feline NSCs are placental feline NSCs.

In a specific embodiment, the NSCs of placentas come from placentas recovered during the gestation period. In another embodiment, the NSCs from placentas come from placentas recovered at the end of the gestation.

### Characteristics of the Feline NSCs

In a specific embodiment, the feline NSCs are characterised by the presence on their surface of the markers CD44 and CD29.

In a specific embodiment, the feline NSCs are characterised by the absence on their surface of the markers CD8, CMH-I and CMH-II.

The analysis of these markers can be carried out by protein and/or transcriptional analysis.

On the transcriptional level, the expression of the markers CD44, CD29, CD8, CMH-I and/or CMH-II can be respectively analysed by measurement of the expression of the genes Cd44, Itgb1, C5ar1 and/or of the family of genes of the type “human leukocyte antigen gene complex” (HLA) HLA-DP, HLA-DM, HLA-DOA, HLA-DOB, HLA-DQ, and HLA-DR or the corresponding homologous feline genes of the type “feline leukocyte antigen gene complex” (FLA). The analysis of the expression of the various genes can be carried out by endpoint PCR, RTqPCR, digital PCR and/or RNA microarray.

On the protein level, the expression of these markers can be studied by the use of antibodies or fragments of antibodies (monoclonal or polyclonal) specifically directed against one or more epitopes of the proteins CD44, CD29, CD8, CMH-I and/or CMH-II. The techniques associated with these antibodies comprise, inter alia, cytometric analysis, western blots, the ELISA test, immunofluorescence and/or immunohistochemistry. Alternatively marker proteins of the ligand or inhibitor type can be used as analysis tools. Other technologies using oligonucleotides marked by a probe can be used to evaluate this expression such as the use of aptameters, RNA probes and/or DNA probes.

Besides their phenotypic characteristics, the feline NSCs can be characterised by their biological characteristics.

In a specific embodiment, the population of feline NSCs is characterised in that at least 80% of the cells of said population have a consecutive cell-doubling capacity greater than 20 cumulative doublings.

“Cell-doubling capacity” means that a population of feline NSCs is capable of doubling in number more than 20 times in total. In particular, the feline NSCs have a cell-doubling capacity of between 20 and 50 cumulative doublings, in particular from 20 to 40, more particularly from 20 to 30. This significant proliferation capacity allows cell amplification of the scale up/scale out type and makes a manufacturing method using this cell population industrialisable. The significant proliferation capacity also means that the population of feline NSCs allows an amplification over several passages ranging from 1 to more than 15 passages.

To evaluate the number of doublings, at each cell passage, the cells are detached from their substrate, centrifuged then counted, for example by the trypan blue exclusion technique using an electronic counter. The doubling number at each cell passage is calculated according to the following formula: Nb of doublings=LOG(Nf/Ni)/LOG(2) (Nf: number of final cells and Ni: number of initial cells). The total number of cell doublings is equal to the sum of the cumulative number of doublings at each cell passage.

Moreover, the mesenchymal stem/stromal cells coming from adult cat tissue can pose limits to the industrialisable nature of the invention because of a possible infection by a virus of the Foamy virus type, the prevalence of which from 20 to 80% of cats, as was demonstrated for the mesenchymal stem cells coming from adipose tissue (8; 9; 10). This virus limits the proliferation of the cells by causing premature cell death during the phase of cell proliferation. Using cells of neonatal origin allows to limit the use of cells infected by this virus and thus to provide an advantage for the industrialisation of the cell culture. It has been demonstrated that the foetus-mother barrier allows a reduction of the transmission of certain pathogens from the mother to the foetus (11).

In a specific embodiment, the feline NSCs have a capacity for adhesion to plastic. “Capacity for adhesion onto a plastic substrate” means that the population of NSCs is characterised by its property of adhesion onto a plastic substrate.

In a specific embodiment, the feline NSCs have a morphology of the fibroblast type. “Morphology of the fibroblast type” means mononuclear cells having a fusiform morphology; that is to say cells elongated in length in culture on a plastic substrate and the ends of which have cytoplasmic extensions. This morphology attests to a capacity of these cells to extend in order to ensure an adhesion of the focal type onto a substrate and cell polarisation. This aspect can be evaluated by studying the maximum and minimum Feret diameter of the cells, the surface occupied by the cells on a 2D substrate and the corresponding aspect ratio (minimum Feret/maximum Feret).

In a specific embodiment, the feline NSCs have an immunomodulating potential. “Immunomodulating potential” means that in a specific embodiment, the population of feline NSCs is characterised in that at least 80% of the cells of said population of feline NSCs have an immunomodulating potential.

This property of immunomodulation can be characterised by the capacity of the feline NSCs to express a set of immunomodulating factors such as PGE2, IL-6, IL-10, TGF-Iβ, IDO, iNOS, HGF, KGF, CCL2 and/or TSG-6. Indeed, in the presence of an inflammatory context, the feline NSCs can modify their phenotype. The inflammatory context can be imitated in vitro by stimulation of the cells via cytokines and/or growth factors such as IFN-γ, IL-1, IL-6 and/or TNF-α. A modification of the phenotype of the feline NSCs manifests itself as the capacity of the feline NSCs to modify the transcriptional and/or protein expression of markers involved in immunomodulation. These markers include PGE2, IL-6, IL-10, TGF-β, IDO, iNOS, HGF, KGF, TSG-6, CCL2, CMH-I, CMH-II, HLA-E.

The immunomodulating potential of the feline NSCs can also be characterised by the antiproliferative effect of the feline NSCs on peripheral blood mononuclear cells (PBMC) treated with a mitogenic agent such as phytohemagglutinin, concanavalin A and/or with lipopolysaccharides. The immunomodulation of the feline NSCs can also be evaluated by the capacity of the feline NSCs to inhibit the proliferation, the secretion of pro-inflammatory cytokines and/or the differentiation of the T, NK lymphocytes, B lymphocytes, of the monocytes and/or macrophages.

In a specific embodiment, the feline NSCs have a capacity for chondrogenic differentiation. “Capacity for chondrogenic differentiation” means that the NSCs have the capacity to be able to differentiate into chondrocytes. The expressions “chondrocyte differentiation” and “differentiation into chondrocytes” can also be used indifferently.

This capacity for differentiation into chondrocytes can be evaluated by the protein and/or transcriptional study of the specific markers of the cartilage such as type 2 collagen (COL2A1), SOX-9 (SOX9), aggrecan (ACAN), cartilage oligomeric matrix protein (COMP), type IX collagen (COL9A1), type XI collagen (COL11A1), type IIB collagen (COL2B) after induction of the chondrogenesis. Moreover, viscoelastic properties of the extracellular matrix, expressed by the differentiated NSCs, close to the viscoelastic properties of the cartilage, can be used as a characteristic for the evaluation of the chondrogenesis.

In a preferred mode of culture, to induce the chondrogenesis, the NSCs are detached from their substrate by trypsinisation and used to form micromasses by gravitation in a drop of amplification medium for example such as DMEM (1 g/L of glucose) supplemented with 10% FBS (vol:vol), 2 mM of glutamine and 0 to 20 ng/ml of fibroblast growth factor (FGFb). A micromass of NSCs is thus formed after 24 h of incubation, at the base of the drop. This micromass is recovered and placed in the presence, for 7 to 28 days, of a chondrocyte differentiation medium composed of DMEM with 4.5 g/L of glucose to which TGF-β3 or a combination of TGF-β1/BMP-2, in particular of 10 ng/ml of TGF-β3 or of 10 g/ml TGF-β1 and 50 ng/ml of BMP-2 is added. At the end, RNA or protein extraction is carried out in order to analyse the expression of specific markers of the chondrogenic lineage such as type II collagen, aggrecan, COMP, SOX9.

In a specific embodiment, the population of feline NSCs is characterised in that at least 80% of the cells of said population of feline NSCs have a potential for osteogenic differentiation. This is also called potential for osteogenic differentiation or osteogenesis.

This potential for osteogenic differentiation can be evaluated by the protein and/or transcriptional study of the specific markers of bone (ALPL, RUNX2 . . . ) or by analysis of the calcium deposits after induction of the osteogenesis for 7 to 15 days. This osteogenic induction can be carried out by monolayer culture of the NSCs in the presence of a medium for osteogenic differentiation containing a corticosteroid, such as dexamethasone (0.1-1 μM), a reducing agent such as ascorbic acid 2-phosphate (between 0 and 200 μg/ml) and (β-glycerophosphate (0-50 mM). BMP-2 can for example replace the dexamethasone.

At the end of the differentiation process, the presence of calcium deposits in the culture dish is revealed by colouring with a solution of Alizarin Red 1% (weight/volume) under a microscope. The presence of deposits reveals the potential for osteogenic differentiation.

Alternatively or in addition, a protein and/or transcriptional study of the specific markers of bone (ALPL, RUNX2) can be carried out.

### Pharmaceutical Composition and Injectable Solution Comprising Feline NSCs

Another aspect of the invention relates to a pharmaceutical composition comprising a population of feline NSCs as described above.

Thus, the present invention also relates to a pharmaceutical composition comprising a population of feline NSCs as described above and a pharmaceutically acceptable carrier.

In a specific embodiment, the feline NSCs of said composition come from a sample of neonatal tissue, in particular from one or more placentas and/or from one or more umbilical cords, or from a sample of neonatal biological fluid, in particular the blood of one or more umbilical cords or the amniotic fluid.

In a specific embodiment, said pharmaceutical composition includes a population of feline NSCs of between 3 million and 20 million cells for 0.5 ml to 10 ml of solution, and more specifically of between 5 and 15 million cells for 1 to 2 ml of solution, and even more specifically 10 million cells for 1.5 ml of solution. Thus, in a specific embodiment, the pharmaceutical composition comprises between 3·10⁵ and 4·10⁷ cells/ml, in particular 2.5·10⁶ and 1.5·10⁷ cells/ml, more particularly 1·10⁷ cells/ml. In a specific embodiment, the pharmaceutical composition comprises between 5 and 15 million feline NSCs, in particular 10 million.

Typically, the pharmaceutically acceptable carrier of this composition and/or the packaging allows to maintain this proportion of viable NSCs for a sufficient time, in a frozen or unfrozen manner according to the following conditions. The carrier can be any type of liquid, gel, solid polymer capable of containing these NSCs without deteriorating their desired properties, in particular a saline aqueous solution, serum, culture medium, a cryopreservation medium. The packaging can be all types of receptacles, containers, medical devices capable of aseptically insulating the pharmaceutical preparations from the outside environment and/or from the transportation environment and/or from the handler. In particular, the packaging allows to maintain the integrity and the formulation of the pharmaceutical composition and/or to facilitate the distribution/transport of the pharmaceutical composition. Moreover, in a specific embodiment, the carrier and/or the packaging can be used to improve the properties of the feline NSCs for their therapeutic use. For illustrative purposes, growth factors, cytokines, active principles can be incorporated into the carrier or bonded to the packaging and thus act on the properties of the feline NSCs in such a way as to improve them. The availability and/or release of these chemical and/or biological agents can be of a kinetic nature.

In a specific embodiment, said pharmaceutically acceptable carrier is a solution comprising a cryoprotectant.

The solution can be any solution allowing the freezing and/or the thawing of the cells while limiting the biological influence of these methods on the cells like inducing cell death, differentiation, inducing cellular senescence, osmotic shocks, inducing membrane porosity, the modification of the membrane composition and/or phenotypic changes.

In a specific embodiment, the solution corresponds to DPBS (Dulbecco's phosphate-buffered saline), DMEM (Dulbecco's Modified Eagle Medium), MEM (Minimum Essential Media), a solution comprising foetal bovine serum (FBS), a solution comprising animal serum, and/or any other isotonic solution.

In a specific embodiment, said solution comprises between 0 and 20% FBS, more particularly between 5 and 20%, even more particularly 10%.

In a specific embodiment, said solution is devoid of product of animal origin. Insofar as the entirety of the formulation of the solution is compatible with an in vivo injection, the cryopreservation solution can be used as an injectable solution for the treatments to which the pharmaceutical composition described in said invention relates.

Cryoprotectant means any compounds allowing to ensure the function of cryopreservation. According to a more specific embodiment, said cryoprotectant is chosen from glycerol, dimethyl sulfoxide (DMSO), propylene glycol, the proteoglycans, trehalose, “bovine serum albumin” (BSA), gelatine, polyethylene glycol (PEG), polyacrylic acid, poly-L-lysine, ethylene glycol or a combination of several of these cryoprotectants.

Commercial solutions comprising a cryoprotectant usable in the pharmaceutical composition are preformulated synthetic cryoprotectant solutions of the type StemAlpha, CryoStor® CS2, CS5 or CS10.

In a specific embodiment, said solution comprises from 0.5 to 30% glycerol, from 0.5 to 30% DMSO, from 0.5 to 30% propylene glycol or from 0.5 to 20% poly-L-lysine. In particular, said solution comprising a cryoprotectant is a solution comprising from 2 to 10% DMSO, more particularly 5% DMSO. In particular, said solution comprising a cryoprotectant is a solution comprising 2 to 20% glycerol.

In a specific embodiment, one or more adjuvants can be added to the pharmaceutical composition such as ammonium chloride, Ringer's lactate or BSA.

In a specific embodiment, said solution comprising a cryoprotectant is a solution of DMEM comprising FBS, in particular from 5 to 20% FBS and more particularly 10%, and comprising from 1 to 90% DMSO, more particularly from 0.5 to 20%, more particularly from 5 to 10%, in particular 5%.

In another specific embodiment, said solution comprising a cryoprotectant is a solution of DMEM comprising FBS, in particular from 5 to 20% FBS and more particularly 10%, and from 2 to 20% glycerol.

In another specific embodiment, the pharmaceutical composition is characterised in that it is in frozen form.

The pharmaceutical composition can be cryogenised with the use of a suitable cryoprotectant, capable of guaranteeing the integrity and the formulation of the pharmaceutical composition. The pharmaceutical composition can thus be preserved at −196° C., for long-term storage (greater than 12 months). To be used, it undergoes thawing, as described below.

In a specific embodiment, said composition is a ready-to-use composition.

“Ready to use” means that the pharmaceutical composition comprising feline NSCs is ready to be injected into an individual. The re-culturing of the cells before use in an individual is not necessary and the cells do not have to be resuspended in a physiological medium, even when they are formulated with a cryoprotectant as described above. The expression “ready to use” means that only a thawing step is necessary when the composition is in frozen form, before injection into the patient.

This pharmaceutical composition that can be frozen is thus usable at any time. The latter has the advantage of making the treatment available in the shortest times while limiting the human intervention necessary for its effectiveness and while limiting the risk of contamination inherent in each manipulation by an operator. Thus, it is possible to separate the method for obtaining the pharmaceutical composition from its final clinical use.

Another aspect of the invention relates to a ready-to-use injectable solution comprising a population of feline NSCs as described above and a cryoprotectant as defined above.

In a specific embodiment, said ready-to-use injectable solution comprises a single dose of 5 to 15 million NSCs, more particularly 10 million, and a solution comprising a cryoprotectant as defined above.

In a specific embodiment, the cryoprotectant in said injectable solution is DMSO. In a specific embodiment, the solution comprises between 0.5 and 30% DMSO, in particular between 2 and 10%, more particularly 5%.

In a specific embodiment, the ready-to-use solution is injected at a volume of between 1 and 5 ml, more particularly 1.5 ml.

### Therapeutic use of the Feline NSCs

The characteristics of the feline NSCs described above allow a use of these cells in cell therapy.

“Cell therapy” means a therapeutic treatment comprising the administration of cells capable of inducing a beneficial therapeutic effect in a feline.

In the context of a regenerative medicine approach, this cell therapy is capable of directly (cellular differentiation) or indirectly (secretion of biological factors, activation or inhibition of cells of the environment) promoting recovery, homeostatic and/or immune equilibrium of one or more tissues or organ in a feline individual waiting for such a treatment.

Thus, in another aspect the present invention relates to a population of feline NSCs, a pharmaceutical composition or an injectable solution as described above for their use in cell therapy.

This use in cell therapy can be an autologous, allogenic, syngeneic or xenogeneic use.

In a specific embodiment, the use in cell therapy is an allogenic therapeutic use, also called heterologous.

In a specific embodiment, the use in cell therapy is a xenogeneic therapeutic use.

In a specific embodiment, the present invention relates to a population of feline NSCs, a pharmaceutical composition or an injectable solution as described above for their use in the treatment of feline stomatitis.

“Stomatitis” means an inflammation of the oral cavity. Stomatitis is designated by different terms according to the etiology, the therapeutic approach and/or the clinical signs observed. For example and non-exhaustively, stomatitis includes gingivitis, periodontitis, caudal stomatitis, buccal stomatitis, chronic stomatitis, lympho-plasmocytic stomatitis or granulomatous stomatitis.

The stomatitis can have an origin that is infectious (bacterial, fungal), autoimmune, inflammatory, subsequent to chemotherapy and/or radiotherapy and/or subsequent to an organic, metabolic drug treatment. In a specific embodiment, the pathological origin of the stomatitis is viral and, non-exhaustively, generated after an infection by feline calicivirus, the herpes virus or by the feline immunodeficiency virus.

In a specific embodiment, the feline stomatitis is a chronic stomatitis, in particular a chronic gingivostomatitis (CGS).

In a specific embodiment, 5·10⁵ to 10·10⁶ cells/kg, more particularly 1·10⁶ to 5·10⁶ cells/kg are administered.

In a specific embodiment, 5·10⁵ to 2·10⁷ feline NSCs, in particular from 5·10⁶ to 1.5·10⁷, more particularly 1·10⁷, or a composition or injectable solution comprising such a dose, are administered.

In a specific embodiment, the subject is treated with the administration of a single dose as defined above. This means that a single dose of 3·10⁶ to 2·10⁷feline NSCs, in particular from 5·10⁶ to 1.5·10⁷, more particularly 1·10⁷, suffices to treat the subject.

“Treat the subject” means that the subject to which feline NSCs as described in the present invention were administered shows a reduction in the symptoms for a duration of at least 2 months, in particular at least 4 months, in particular at least 6 months. The condition of the subject is thus improved by the treatment. This also means that the subject to which the feline NSCs were administered shows an improvement in its condition in the sense that this allows a reduction of the frequency and/or of the dose, or the total elimination of the need for other conventional therapeutic or analgesic solutions known to a person skilled in the art.

“Conventional therapeutic or analgesic solutions known to a person skilled in the art” means non-exhaustively total or partial dental avulsion, ciclosporin, steroidal or non-steroidal anti-inflammatory drugs, antibiotics, interferon omega (IFN), corticoids, gabapentin, morphine and/or buprenorphine.

In a specific embodiment, the administration to the subject is carried out at least once to treat said subject.

In certain cases, several administrations to the subject over time are carried out.

In a specific embodiment, at least two administrations to the subject are carried out, two administrations being spaced apart by at least 2 months from one another, in particular spaced apart by 2 months to 5 years, by 2 months to 4 years, by 2 months to 3 years, by 2 months to 2 years, by 2 months to 1 year, or by 2 months to 6 months.

The NSCs can be administered locally or in a specific embodiment intravenously (IV). IV administration means administration of the NSCs directly into the venous system of the animal using a catheter or a needle or for example via a bag of perfusion solution or for example via the nozzle of the intravenous set with a particular administration speed. Administration speed means a flow rate of perfusion in a specific embodiment of 2·5·10⁴ to 1·10⁶ NSCs/min. For example the NSCs can be administered at the speed of 5·10⁵ NSCs/min.

Preferably, the administration is carried out intravenously, for example using a perfusion.

In a specific embodiment, said population of feline NSCs or said pharmaceutical composition for the use as described above is characterised in that the administration is carried out in the form of a single dose of 5·10⁵ to 2·10⁷ feline NSCs, in particular from 5·10⁶ to 1.5·10⁷, more particularly 1·10⁷, to treat the subject, in particular intravenously.

In a specific embodiment, this use can be combined with other types of treatment/interventions. In particular, it can be carried out after a total dental avulsion, and/or during or after a drug treatment or during anaesthesia.

In a specific embodiment, this use can be carried out in subjects not having undergone dental avulsion or in subjects having undergone a partial dental avulsion.

“Partial dental avulsion” means any surgical operation aiming to extract between 1 and 29 teeth, more particularly between 1 and 26 teeth, more particularly between 1 and 16 teeth. In an alternative operating mode, a partial avulsion can represent only the extraction of the teeth specifically affected by the oral lesions or the extraction of all the teeth except the canines.

In a specific embodiment, the present invention relates to the feline NSCs, the pharmaceutical composition or the injectable solution as described above for their use in the treatment of feline stomatitis, in combination with the administration of one or more anti-inflammatory, antalgic, immunomodulating, anti-infective and/or antiviral treatments related to the stomatitis or not.

In a specific embodiment, the present invention relates to the feline NSCs, the pharmaceutical composition or the injectable solution as described above for their use in the treatment of stomatitis in felines, in combination with the administration of an anti-inflammatory.

This anti-inflammatory can be a steroidal or non-steroidal anti-inflammatory.

The steroidal anti-inflammatory drugs include glucocorticoids and corticosteroids. The non-steroidal anti-inflammatory drugs include buprenorphine, aspirin, ibuprofen, ketoprofen, methylprednisolone acetate. The therapeutics with an anti-inflammatory action include laser therapy. The antalgics include the morphinic drugs and alpha-2 agonists. The immunomodulators include cyclosporines and rapamycin. The anti-infectives and antivirals include antibiotics and recombinant interferons.

In a specific embodiment, the feline NSCs are administered for the treatment of feline stomatitis in subjects refractory to one or more of the therapeutic or analgesic solutions mentioned above.

“Refractory” means any medical care for feline CGS that is not satisfactory, which has a failure in effectiveness, a recurrence of clinical signs, which generates undesired side effects, which generates an intolerance or an adverse immune reaction, inducing a failure in compliance, requiring the use of a drug dose that is too high with respect to a conventional treatment and/or the fact of having to use polypharmacy that is too complex and unsuitable.

The use of the feline NSCs according to the invention allows at least a clinical improvement or a complete remission in more than 50%, more particularly 70%, more particularly 80% of the animals suffering from CGS, having undergone a total dental avulsion and/or refractory to conventional therapies. In particular, this change is observed without previous selection of the cats based on their blood phenotype and more particularly without selection of the proportion of cells having a low level of expression of the marker CD8 (CD8+low) in a population expressing this marker (CD8+global).

The effectiveness of the treatment can be evaluated in various ways. In a specific embodiment, the effectiveness of the treatment is measured by using an evaluation system established by a veterinarian, based on the activity index of the CGS (The Stomatitis Disease Activity Index: SDAI). The SDAI score takes into account the following parameters: oral inflammation (maxillary and mandibular), gingival inflammation (maxillary and mandibular), inflammation at the palatoglossal arch, inflammation of the salivary gland, oropharyngeal inflammation, lingual or sublingual inflammation. The evaluation of the SDAI can be carried out upon inclusion then at various times after the perfusion or the injection of the pharmaceutical preparation.

Clinical improvement means an improvement in the clinical score (SDAI) expressed in the form of a recovery percentage. The recovery percentage is obtained by the formula {1-(SDAI_{f}/SDAIᵢ)}×100 where SDAI_{f} corresponds to the final SDAI obtained, and SDAIᵢ corresponds to the initial SDAI obtained. Alternatively, the effectiveness of the treatment can be assessed over an interval of time by the calculation of a recovery percentage using, as an analysis variable, an SDAI at a given intermediate time (SDAIₜ) instead of an SDAI_{f} and a formula of the type {1-(SDAIₜSDAIᵢ)}×100.

In a specific embodiment, a percentage greater than or equal to 15% is considered to be a clinical improvement not caused by a placebo effect (13). Given this response threshold, the pharmaceutical composition according to the invention allows a clinical improvement in 83.3% of cases at 2 months (n=5 cats); in 83.3% of cases at 3 months (n=5 cats); in 100% of cases at 6 months (n=6 cats) post-treatment.

Clinical remission means the animals with an SDAI score of less than 2 at the time of clinical evaluation (Dental Vets, 2015). In a specific embodiment, clinical remission is also when the recovery percentage as described above is greater than or equal to 90% between the final evaluation (SDAI_{f}) and the initial evaluation (SDAIᵢ).

In another specific embodiment, the effectiveness of the treatment can be evaluated by monitoring the changes in the symptoms related to the disease. The treatment is thus considered to be effective if it allows to eliminate the need for the other conventional therapeutic or analgesic solutions known to a person skilled in the art or to reduce the frequency of use or the dose thereof, if it contributes to reducing the eating disorders (dysorexia) of the animals having a symptom of undernutrition, if it allows a gain in activity and a gain of weight in the subjects treated having an initial loss of activity, if it allows to reduce the intensity of the pain caused by the pathological state of the subject. All or part of these parameters can be monitored by the owners in monitoring forms (Example 2).

### Method for Treating Feline Stomatitis

The present invention also relates to a method for treating feline stomatitis comprising the administration of feline NSCs, of a pharmaceutical composition or of an injectable solution comprising feline NSCs as described above, to a subject.

All of the embodiments described above relating to the feline NSCs, a pharmaceutical composition or an injectable solution comprising said cells for their use in cell therapy apply to the treatment method.

### Method for Obtaining a Composition Comprising Feline NSCs

Another aspect of the invention relates to an in vitro method for obtaining a pharmaceutical copolymer according to the invention.

Thus, the present invention also relates to an in vitro method for obtaining a ready-to-use pharmaceutical composition comprising feline NSCs as described above, said method comprising the following steps:

a. a step of providing one or more feline neonatal biological samples, the biological sample(s) having been previously obtained from one or more individuals;
b. a step of isolating the population of feline NSCs present in the biological sample(s);
c. optionally, the ex vivo amplification of the NSCs obtained in step b.;
d. at least one step of cryopreservation of the population of feline NSCs obtained after step b. and/or c., to form a cell bank;
e. a step of thawing the population of feline NSCs after step d.;
f. optionally, the ex vivo amplification of the NSCs after step e.;
g. optionally a step of stimulation by a physical chemical and/or biological effector of the feline NSCs to increase their biological and therapeutic properties, during step f.;
h. at least one step of verifying the biological properties of the feline NSCs after step b., c., d., e.,
f., and/or g.;
i. a step of cryopreservation of the population of feline NSCs obtained with an acceptable carrier of administration to the patient.

The feline neonatal biological samples from which the feline NSCs can come were described above.

“Isolation” means the means implemented to extract the cells from their original source of tissular and/or biological fluid nature. Typically these means correspond to the mechanical dissection of the tissue allowing to obtain tissue fractions usable in a laboratory. These tissue fractions are then cultured for an isolation of the explanation type, thus using the migration capacities of the resident cells. Another isolation means is for example the use of digestion enzymes leading to the catabolism of the extracellular matrix and the release of the cells over a given time of 10 min to 16 h under a controlled temperature. Among these digestion enzymes it is possible to mention non-exhaustively the collagenases, pronase, hyaluronidases and/or other matrix proteases. Another isolation means, for the biological fluid sources and/or cell suspensions obtained from enzymatic digestion of tissue, involves the use of purification of cells by centrifugation on a density gradient of the Ficoll, Percol and/or glucose type. The methods mentioned above can be used alone or in combination. The result of these isolation methods must favour the isolation of the NSCs independently of the hematopoietic cells, blood cells or other contaminant cell types not corresponding to NSCs or NSCs not having the biological properties mentioned above.

In order to obtain greater quantities of feline NSCs, a step of amplification of the cells by adhesion to plastic can be carried out. In a specific embodiment, a plastic substrate treated or not for cell adhesion, and/or in the presence or not of fibroblast growth factor (FGF, FGF-2, FGFb), dexamethasone and/or ascorbic acid 2-phosphate (A2P), and in a cell culture medium correctly defined by a person skilled in the art, is used.

“Amplification step” means any step allowing a proliferation of the NSCs on a plastic or polymer substrate. This phase must be capable of favouring the presence of the NSCs to the detriment of other cell types not satisfying the characteristics of the NSCs. It must also ensure an optimal proliferation of the cells while limiting the phenomena of dedifferentiation, differentiation and/or senescence. This step involves conditions in a controlled atmosphere like a person skilled in the art is capable of establishing for example such as with 90% humidity and including 5% CO₂. The amplification temperature must be constant and between 35-40° C., more precisely between 37-39° C. The culture media, non-exhaustively, include the media of the type Alpha-MEM, DMEM, RPMI, IMDM, Opti-MEM, EGM, EGM-2, synthetic media adapted to the culture of MSCs devoid of endotoxin and/or of serum, synthetic media adapted to good manufacturing practices, supplemented or not by foetal bovine serum (FBS) from 0.1% to 20%, platelet lysate, insulin-transferrin-elenium, defined commercial supplements and/or other growth factors and/or molecules favouring the proliferation of the NSCs while limiting their senescence such as FGF, EGF, VEGF, dexamethasone and/or A2P.

This amplification phase can be carried out on various substrates once the population of NSCs is obtained after the isolation step. These various substrates can be of a 2D or 3D nature.

“Amplification on a 2D substrate” means all methods of cell culture allowing an amplification of the feline NSCs on a monolayer substrate and correctly developed by a person skilled in the art. In specific embodiments the cells can be cultivated in culture dishes made of plastic and treated or not to favour cell adhesion, of the flask type, with one or more levels and/or of the multilayer type with or without continuous perfusion, with or without optimisation of the flow of air.

“Amplification on a 3D substrate” means all techniques known to a person skilled in the art using biomaterials, microcarriers and/or polymers capable of ensuring an amplification of the NSCs in a bioreactor and correctly developed by a person skilled in the art. In specific embodiments, the NSCs can be amplified in bioreactors with stirring, axial and/or vertical, with wave stirring, with rotating bed stirring, in static and/or perfused bioreactors. The biomaterials and/or microcarriers can be of several natures and according to specific embodiments can have a size between 50-500 μm in diameter, have a porosity of a different nature, have a treated surface, negatively or positively charged or not, include growth factors or recombinant proteins of the type integrins and/or extracellular matrix or any other biological/chemical molecules favouring cell adhesion and/or cell proliferation.

In order to carry out the proliferation phase, a cell passage of the NSCs can be necessary and carried out by a method correctly developed by a person skilled in the art. These cell passages can be carried out by detachment of the cells under the effect of mechanical action, of enzymes and/or inhibitors such as, non-exhaustively, trypsin, EDTA and/or recombinant or animal accutase. Moreover, it is possible to carry out these cell passages by the use of biomaterials/microcarriers dissolvable according to a method developed by a person skilled in the art. It should be noted that during the succession of passages, a clonal selection can be carried out during the first passages from P1 to P4 like (11) and lead to a relatively stable population such that the clonogenicity of the population of NSCs only slightly changes. Moreover, these first passages can allow the isolation of a population of NSCs devoid of other contaminant cell types such as lymphocytes and other blood, macrophage, neutrophile, polynuclear cells, endothelial cells and/or fibroblasts.

In a specific embodiment, for the amplification phase, the cells are for example treated with trypsin-EDTA, then replaced in an amplification medium and centrifuged. After replacement in amplification medium, they are inoculated at a rate of 1500 to 5000 cells/cm² or 3D equivalent and cultivated in amplification medium with or without monitoring of the microenvironment and/or culture atmosphere.

During the method, the feline NSCs obtained are suspended in a pharmaceutically acceptable carrier as defined above. In a specific embodiment, the pharmaceutically acceptable carrier is a solution comprising a cryoprotectant.

During the method, it is possible to carry out a step of cryopreservation, in order to create a cell bank such that it can be defined in the industry and established by a person skilled in the art. “Cryopreservation” means the step of freezing the cells and storing for a time ranging from 1 day to 5 years or more. The cell bank is packaged in such a way as to guarantee the integrity and the biological properties of the cell populations. The banks can be of several natures and play several roles. These non-exhaustively include the cell banks of reserves (master bank or seed unit), working banks, legal banks, retention banks and/or bank of ready-to-use therapeutic units for therapeutic uses. It should be noted that the latter bank of therapeutic units can correspond to a pharmaceutical preparation described above.

The step of cryopreservation is carried out after the suspension of the NSCs in a solution comprising a cryoprotectant.

In a specific embodiment, this step of freezing or cryopreservation corresponds to a progressive lowering of the temperature of the cell suspension (−1° C./minute) to reach the storage temperature ranging from −70° C. to −195° C. In an alternative embodiment, the cells can be frozen at a freezing speed between −0.3° C./minute and −99° C./minute. The same freezing protocol can comprise one or more different freezing speeds like in the case of a gradual increase of freezing speed. In an alternative embodiment, the cells are directly frozen without temperature control in a storage unit, the temperature of which is between −70° C. and 195° C. The final storage can be in liquid phase (of the liquid nitrogen type) or gaseous phase (of the type −140° C., −80° C. chamber or storage in nitrogen in gas phase).

In order to make the cryopreserved cells available for therapeutic administration to the subject, a step of thawing is carried out after the freezing step, in the case in which the NSCs used for the administration are in the form of bank units. This thawing is carried out in such a way as to go from the stage of frozen cells to the stage of thawed cells during an embodiment limiting cell death by desiccation, mechanical lesion of the plasma membrane, osmotic shock. In a specific embodiment, the units of NSCs are heated by manual friction for less than 10 min. In another specific embodiment, the units of NSCs are placed in a liquid or dry bain-marie, set to a temperature of between 30 and 40° C. for less than 10 min, in particular to 37° C. for 3 to 5 min. In another specific embodiment, the units of NSCs are placed in an automatic thawing apparatus. In another specific embodiment, the units of NSCs are thawed at ambient temperature for less than 10 min if the cryoprotectant used allows it.

If necessary, for the banks other than cells of ready-to-use therapeutic units, the thawed cells can be reamplified. After thawing, the cells are then resuspended in washing medium (DPBS, DMEM or DMEM+FBS) or in cell amplification medium. In a specific embodiment, the volume of suspension is progressively adjusted in order to limit the osmotic shocks that the cells can undergo. The cells are then re-cultured in order to carry out a new phase of amplification.

Optionally, the feline NSCs can undergo a step of exogenous stimulation/modification during the amplification phase via physical, biological and/or chemical effectors. Exogenous stimulation (also called priming) means all stimulations/modifications of the cells and/or of their microenvironment triggering in the cells a phenotypic change favouring their biological properties in the context of specific therapies. For example, a treatment with cytokines in concentrations between 1 and 500 ng/ml of IFN-γ, of IL-1β, I1-6 and/or of TNF-α allow to significantly increase the expression of molecules performing an immunomodulating activity. In another example mechanical stimulation and/or the induction of chondrogenic predifferentiation can allow to favour the properties of in vitro tissue reconstruction.

In the method, at least one step of verifying the biological properties of the isolated feline NSCs is carried out. In another specific embodiment, the presence of the markers CD29 and CD44 and the absence of the markers CD8, CMH-I and CMH-II are verified. In a specific embodiment, the capacity for cell proliferation comprised of at least 20 consecutive cell doublings, as described above, is verified. In a specific embodiment, the capacity for chondrogenic differentiation of the feline NSCs is verified. These specific embodiments can be combined with each other.

### BRIEF DESCRIPTION OF THE DRAWINGS

FIG. 1A shows the analysis of the feline NSCs by flow cytometry according to the parameters FSC-A/FSC-H allowing to select the unique cells. An analysis according to the parameters FSC-A/SSC-A allows to then select the NSC population of interest.

FIG. 1B, 1C, 1D, 1E, 1F show the evaluation of the expression of the marker of interest by the population of feline NSCs by analysis of the fluorescence for the corresponding fluorochrome. The graph shows the results of the surface markers (in light grey, the specific marker of interest; in dark grey the corresponding isotype). The positivity threshold is placed using the cells marked by the corresponding isotype. The table indicates the number of events analysed, as well as the median of fluorescence of the population of NSCs.

FIG. 1B shows the positivity of the expression of the surface marker CD44.

FIG. 1C shows the positivity of the expression of the surface marker CD8.

FIG. 1D shows the positivity of the expression of the surface marker CMH-II.

FIG. 1E shows the positivity of the expression of the surface marker CD29.

FIG. 1F shows the positivity of the expression of the surface marker CMH-I.

FIG. 1G shows the analysis of the PBMCs by flow cytometry according to the parameters FSC-A/FSC-H allowing to select the unique cells. An analysis according to the parameters FSC-A/SSC-A allows to then select lymphocytes.

FIG. 1H and 1I show the evaluation of the expression of the marker of interest by the lymphocytes, by analysis of the fluorescence for the corresponding fluorochrome. The graph shows the results of the surface markers (in light grey, the specific marker of interest; in dark grey the corresponding isotype). The positivity threshold is placed using the cells marked by the corresponding isotype. The table indicates the number of events analysed, as well as the median of fluorescence of the lymphocytes.

FIG. 1H shows the validation of the specificity of the marker CMH-II on cat lymphocytes.

FIG. 1I shows the validation of the specificity of the marker CMH-I on cat lymphocytes.

FIG. 2 shows the proliferative activity illustrated by the accumulation of the number of cell doublings during the passages.

FIG. 3 shows a photograph representative of the MSCs cultivated 2 weeks in an osteogenic differentiation medium and marked with Alizarin Red. The presence of calcium deposits is highlighted by the red colouring.

FIG. 4 shows a monitoring form completed by the owners of the animals included in the study.

FIG. 5 shows a photograph of the mouth of a cat having undergone dental avulsion and treated with the feline NSCs. This photograph shows the reduction in the oral lesions visible before (photograph on the left) and after treatment (photograph on the right).

FIG. 6 shows the change in the SDAI score of the cats having undergone dental avulsion and treated by the feline NSCs over time (days after perfusion). The median is represented by a horizontal line at the centre of the box plots.

FIG. 7 shows the change in the SDAI score over time presented for each cat included and monitored having undergone dental avulsion and treated by the feline NSCs.

FIG. 8 shows the clinical recovery rate calculated at each monitoring visit (D60, D90, D180) for the 7 cats having undergone dental avulsion and treated by the feline NSCs of the study.

FIG. 9 shows the owner score (/12) evaluated over time (injection D0, 15 days, 2 months, 3 months and 6 months) for each cat having undergone dental avulsion and treated by the feline NSCs.

FIG. 10 shows a photograph of the mouth of a cat having undergone partial dental avulsion and treated by the feline NSCs. This photograph shows the reduction in the visible oral lesions 15 days after treatment (photograph on the left) and 1 month after treatment (photograph on the right).

FIG. 11 shows the change in the SDAI score of the cats having undergone a partial dental avulsion and treated by the feline NSCs over time (days after perfusion).

FIG. 12 shows the clinical recovery rate calculated at each monitoring visit (D60, D90, D180) for the 2 cats having undergone a partial dental avulsion and treated by the feline NSCs of the study.

FIG. 13 shows the owner score (/12) evaluated over time (day of the perfusion, 15 days, 2 months, 3 months and 6 months) for each cat having undergone a partial dental avulsion and treated by the feline NSCs.

### EXAMPLES

### Example 1: Obtaining and Characterising Feline Neonatal Stromal Cells (NSCs)

### A. Method for Obtaining a Population of Feline NSCs

### Collecting Feline Embryonic Membranes

The feline extra-embryonic membranes (placenta, umbilical cord) are removed aseptically during elective ovariohysterectomy carried out on pregnant cats. In the context of an ovariohysterectomy or hysterectomy, the uterus plus or minus the clamped ovarian ducts are deposited in a transport box containing a buffered saline solution for example such as Dulbecco's phosphate (D-PBS) to be transported to the laboratory at a controlled temperature (4-12° C.).

### Isolation of the Feline NSCs from Extra-Embryonic Tissue

The treatment of the extra-embryonic tissue is carried out at most in the 72 h following the removal. All of the steps of treating the tissue are carried out in a controlled environment, in a microbiological safety cabinet (BSC). The tissue is removed aseptically from its transport box and transferred to a Petri dish of 100 cm². The amniotic sac containing the foetus is eliminated by dissection. The placenta having a red colour is transferred into a sterile beaker. The tissue is washed 3 to 5 times in successive baths of D-PBS. The placental tissue is dissected into fragments of approximately 10-20 mm² then subjected to enzymatic digestion while incubating the tissue fragments in a solution composed of DMEM (Dulbecco's Modified Eagle Medium) containing 0.5-4 mg/ml of type I collagenase, and more specifically a concentration of 1 mg/ml of type I collagenase. The enzymatic digestion is carried out at 37° C. for 45 min but a digestion between 15 min and 16 h can be carried out by reducing the incubation temperature (ambient temperature (18-22° C. or 4° C.). At the end of the digestion, the enzymatic activity is stopped by dilution, by adding DMEM containing at least 10% foetal bovine serum (FBS) in an equivalent quantity to the solution for enzymatic digestion. The solution is then filtered over a 70-100 μm screen. The cells recovered are centrifuged at 800 g for 10 min. The cell pellet containing the neonatal stromal cells is rinsed in DMEM and again centrifuged at 800 g for 10 min. The cell pellet is replaced in culture medium consisting of DMEM, 10% FBS, 2 mM glutamine and from 0 to 20 ng/ml of fibroblast growth factor (FGF). The cells are counted and inoculated in culture dishes at a density of between 10⁴ and 2·10⁴ cells/cm². The cells are then cultivated in the culture medium described above in a controlled atmosphere at 37° C. and containing 5% CO₂. The medium is changed after 48 h then every 2-3 days. The cells are passaged when the confluence reaches 70-80%.

### Isolation of the Feline NSCs from Amniotic Fluid

The Treatment of the Amniotic Fluid

is carried out at most in the 24 h following the removal. All of the steps of treating the tissue are carried out in a controlled environment, in a microbiological safety cabinet (BSC). The clamped or sutured uterus, to avoid microbiological contaminations, is transferred under BSC to a 100 mm petri dish. The amniotic sac is separated from the uterus and transferred to a new 100 mm petri dish. The integral sac thus still containing the foetus is pierced using a chisel or scalpel to free the amniotic fluid. The amniotic sac and the foetus are transferred into another dish and the liquid is collected. Optionally, the amniotic fluids of several amniotic sacs can be combined in order to increase the volume of liquid to be treated. Culture medium consisting of DMEM, 10% FBS, 2 mM glutamine and from 0 to 20 ng/ml fibroblast growth factor (FGF) is added to the amniotic fluid in a minimum amount of 50% of the volume removed. Optionally, the suspension can be rinsed with DPBS and centrifuged at 800 g for 10 min. The volume of liquid or of cells re-suspended in an equivalent volume in culture medium is disposed directly in a culture dish in an amount of 100 to 3000 per cm². The culture dishes are thus cultivated in the culture medium described above in a controlled atmosphere at 37° C. and containing 5% CO₂. The medium is changed after 48 h then every 2-3 days. After 1-2 weeks of culture, the colonies of feline NSCs are passaged to allow cell amplification.

### Cell Passage and Amplification

At sub-confluence, the cells undergo cell passage and optionally an amplification procedure. The feline NSCs are rinsed with D-PBS and treated with 0.05% trypsin-EDTA for 2-5 min at 37° C. This allows to detach the cells and form a population of isolated cells. The cells are then replaced in amplification medium consisting of DMEM, 10% FBS, 2 mM glutamine and from 0 to 20 ng/ml fibroblast growth factor (FGF) and centrifuged between 300-500 g for 5 to 10 min. The feline NSCs are replaced in amplification medium and counted by manual counting (trypan blue and Malassez cells) or using an electronic counter. They are then inoculated at a rate of 1500 to 5000 cells/cm² and cultivated on a plastic cell culture substrate in amplification medium and in a controlled atmosphere at 37° C. and containing 5% CO₂. During the process of amplification the cells can undergo between 0 and 15 cell passages.

### Freezing and Cryopreservation of the Feline NSCs

After the first or second cell passage (P1-P2), the cells can be cryopreserved in seed units. To do this, after counting, the feline NSCs are centrifuged between 300-500 g for 5 to 10 min and the cell pellet is replaced in freezing medium composed either of DMEM medium enriched with 5-20% FBS and 5-10% (vol:vol) DMSO or in a commercial cryopreservation medium, containing a fraction of DMSO or not. The cell concentration is between 1·10⁶ and 15·10⁶ cells per ml of freezing medium. The freezing of the cells is carried out in conditions of controlled temperature decrease, by using for example a CoolCell® Cell Freezing Containers (BioCision) container and by following the freezing procedure as it is described by the manufacturer. The cells are then transferred for storage in negative cold at temperatures such as −196° C. for long-term storage (greater than 1 year).

The seed units are used to generate cell units for therapeutic purposes. The seed units are thawed to 37° C. for 3-6 min and amplified in vitro. The cells are inoculated in culture medium at the density of 1500-3000 cells/cm². The cells are amplified by successive passage in vitro. When a significant number of cells is produced (for example >150·10⁶ cells), the cells are frozen according to the protocol described above. The cells are distributed in hermetically sealed sealable flasks in an amount of 1·10⁶-1.5·10⁷ cells/ml in freezing medium free of product of animal origin (for example such as the cryopreservation medium Recovery™ Cell culture freezing medium (Thermo Fisher), Cryostem™ freezing medium (Biological Industries), Cryostor™ (Biolife Solution). The flasks are decreased in temperature according to a protocol of controlled temperature decrease, at a rate of −1° C./min to −80° C. The flasks are then transferred at −80° C. for storage for a maximum time of 24 months.

### B. Characterisation of the Population of Feline NSCs

In order to evaluate the purity and the functionality of the feline NSCs isolated from feline placentas, tests are carried out on aliquots of cells currently being amplified or after freezing/thawing of a seed unit or of a therapeutic unit.

### Cytometric Analysis of the Feline NSCs

The expression of surface markers of the feline NSCs is carried out by flow cytometry by using the following anti-feline antibodies: CD8 (vpg9; Biorad), CMH2 (vpg3; Biorad); and the antibodies specific to other species and crossing with the cat: CD44 (IM7; Biolegend); CD29 (TS2/16; Biolegend). For the unconjugated antibody anti-CMH2, a secondary mouse anti-immunoglobulin antibody (IgG) coupled with allophycocyanin (APC) (eBiosciences) is used afterwards. Isotypic controls are used to set the background noise for each fluorochrome used: anti-mouse IgG2a (COL2002/COLI205C; Monoclonal Antibody Center); anti-mouse IgG1 coupled with PE (MOPC-21; Biolegend); anti-rat IgG2b coupled with APC (IM7, Biolegend).

In summary, the feline NSCs in culture are detached from their plastic substrate by trypsinisation and are rinsed with D-PBS. The cells are distributed into tubes at a rate of 10⁵−5·10⁵ cells/tube. The cells are centrifuged (500 g/5 min) and replaced in 25-1000 μl of marking buffer (D-PBS and from 0.5-1% (v/v) of bovine serum albumin (BSA) or from 0.5-2% (v/v) foetal bovine serum). The primary antibody coupled or not with a fluorochrome and specifically targeting the membrane marker of interest is added. The optimal concentration of antibody used for the marking must be previously determined by a person skilled in the art. The incubation necessary for the marking must also be determined by a person skilled in the art and be between 15 min and 10 h at 4° C. away from light. In particular, the cells are incubated 30 min at 4° C. away from light. Marking with a secondary antibody targeting the primary antibody can be carried out, after washing with D-PBS, in the case in which the primary antibody is not directly coupled with a fluorochrome. After each incubation with an antibody (primary and secondary) or the corresponding isotype, the cells are washed 2 times in 1 ml of D-PBS and centrifuged (500 g/5 min). The cell pellets are replaced in a volume of 100-500 μl of marking buffer for reading with a flow cytometer (Accuri C6, BD Biosciences).

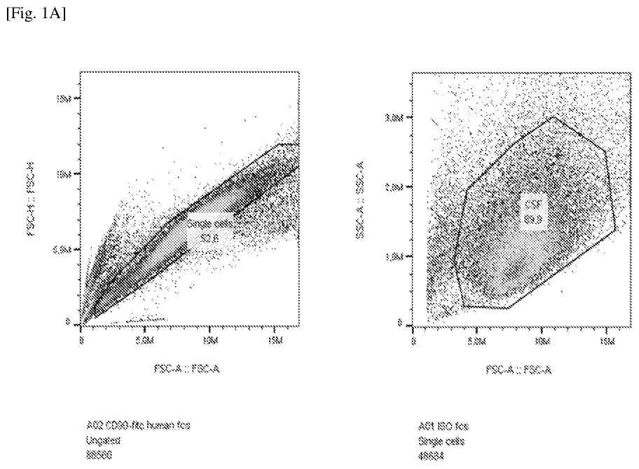

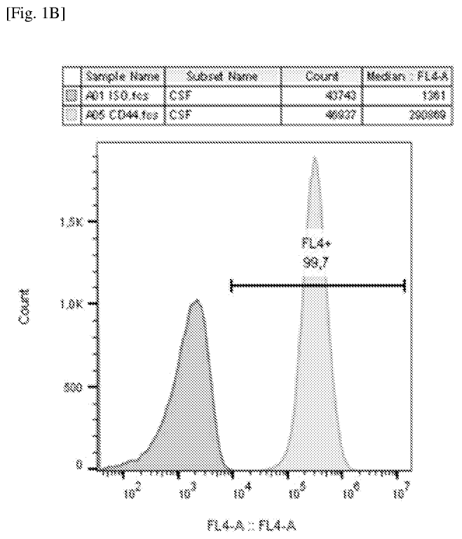

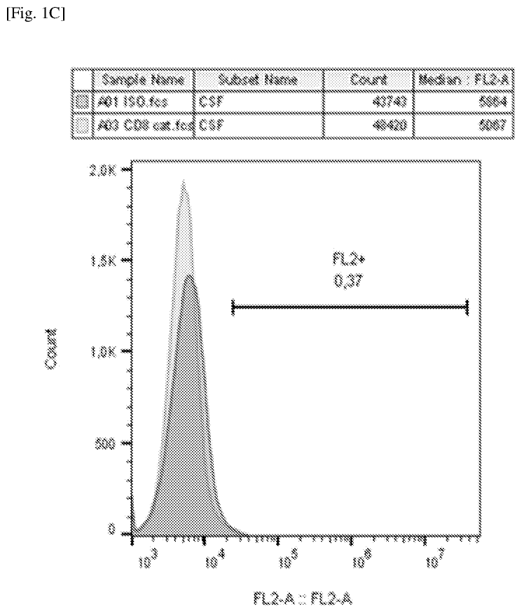

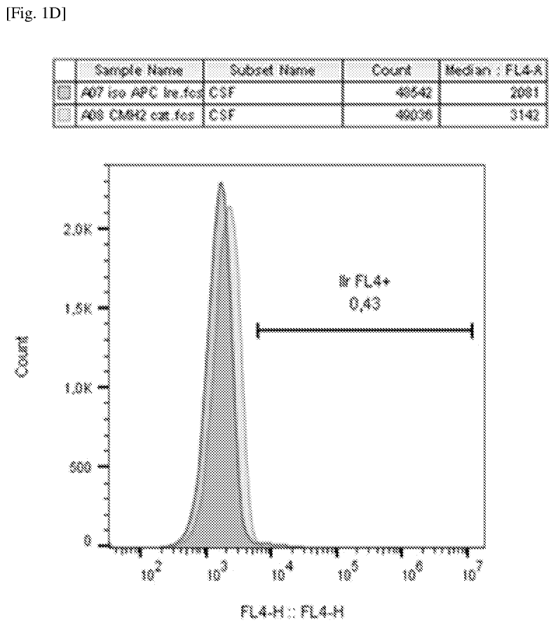

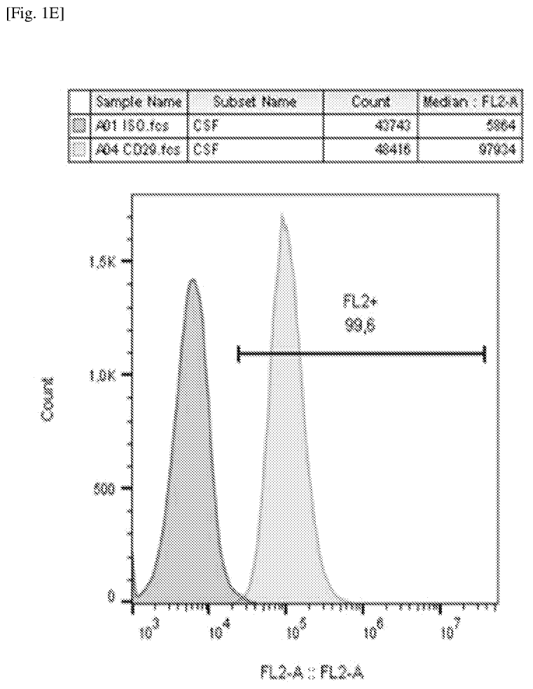

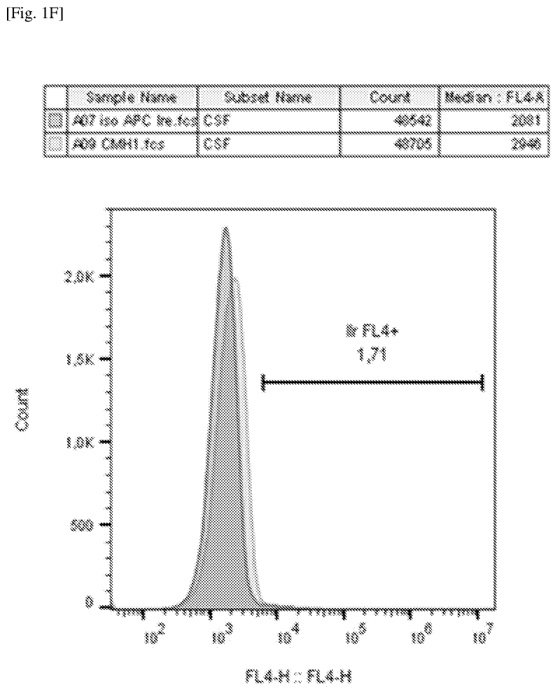

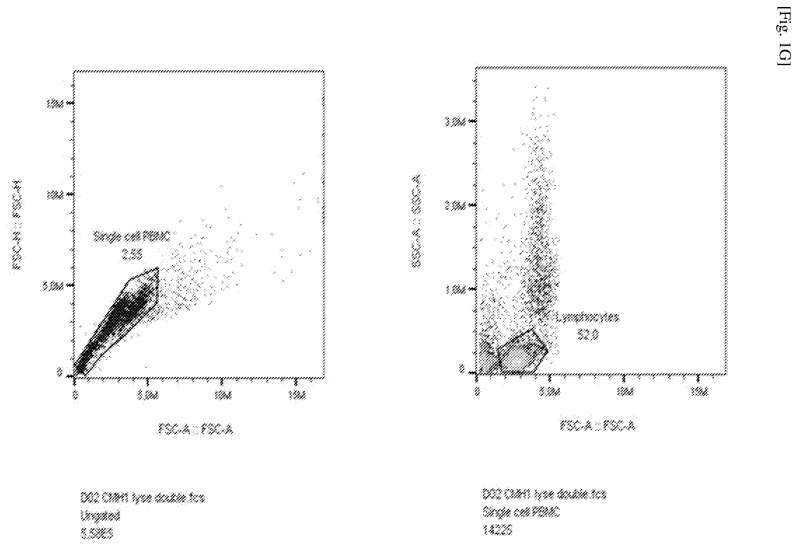

FIGS. 1A to 1I show an example representative of the results of cytometric analysis of the feline NSCs.

Thus, the presence of the markers CD44 and CD29 is revealed, and the absence of the markers CMH-II and CD8 on the surface of the feline NSCs according to the invention.

### Evaluation of the Proliferative Activity of the Feline NSCs

The proliferation of the feline NSCs is evaluated during 6 to 15 consecutive cell passages to determine the proliferative activity of the cells. At each cell passage (1 time per week; or every 6 to 8 days), the cells are detached from their culture substrate using trypsin/EDTA 0.5% for 2-3 min. Culture medium is added and the cells are centrifuged 5 min/300 g. The cell pellet is replaced in a defined volume of culture medium and the cells are counted by a trypan blue exclusion technique using an electronic counter. The doubling number at each cell passage is calculated according to the following formula: Nb of doublings=LOG (Nf/Ni)/LOG (2) (Nf: number of final cells and Ni: number of initial cells). The total number of cell doublings is equal to the sum of the number of cumulative doublings at each cell passage.

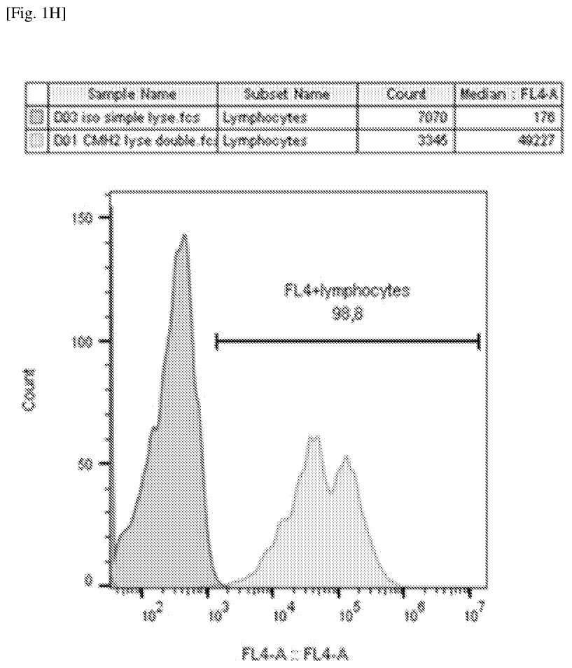

FIG. 2 illustrates the number of cumulative doublings over 8 cell passages of a line of feline NSCs.

Thus, the feline NSCs are capable of 30 consecutive doublings in the space of 7 weeks (at a rate of one passage per week).

### Potential for Osteogenic Differentiation of the Feline NSCs

The feline NSCs are detached from the plastic substrate by trypsinisation and counted. The feline NSCs are inoculated at a density of between 1500 and 5000 cells/cm² in a 6-well plate in amplification medium under a controlled atmosphere at 37° C. and containing 5% CO₂. When the cells reach 50-75% confluence, the proliferation medium is removed and replaced with osteogenic differentiation medium composed of DMEM, 10% FBS, 2 mM glutamine, 0.1 μM dexamethasone (Sigma), 50 μM ascorbic acid 2-phosphate (Sigma) and 10 mM (β-glycerophosphate (Sigma). The medium is renewed 2 times/week, for a period of between 10 and 15 days.

At the end of the differentiation process, the wells are washed with D-PBS and the cells are fastened with for example a solution of 10% neutral buffered formalin for 1 h at minimum. Colouring with a solution of 1% (weight/volume) Alizarin Red is carried out to reveal the presence of calcium deposits. The wells are then rinsed in H₂O and the colouring is analysed under a microscope. The presence of calcium deposits allows to conclude that there is a capacity to differentiate in the osteogenic line.

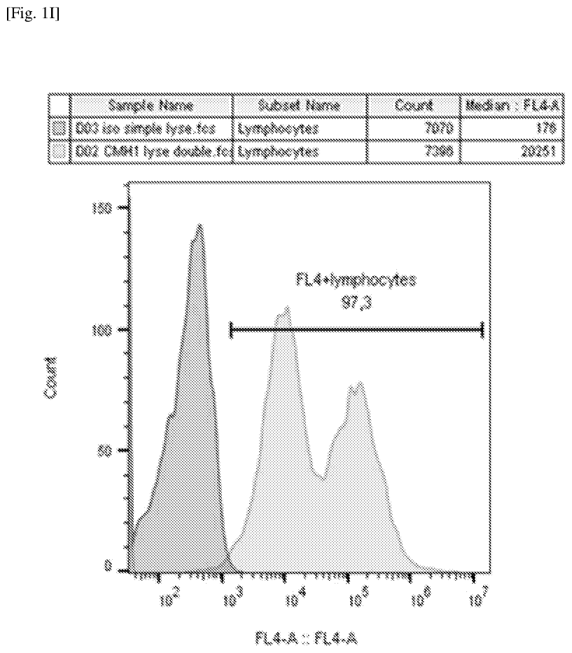

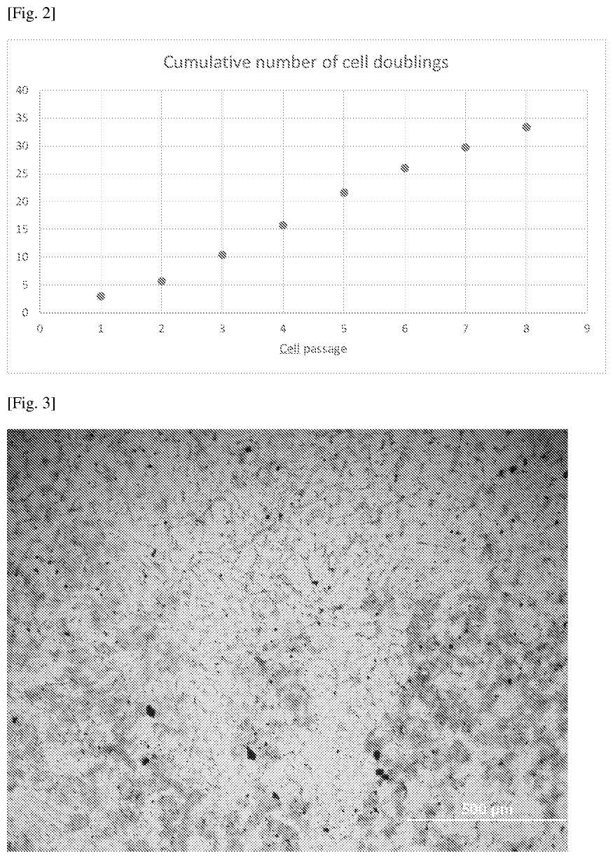

FIG. 3 shows an example of feline NSCs cultivated 14 days in osteogenic differentiation medium and coloured with Alizarin Red.

The presence of calcium deposits demonstrates that the feline NSCs have a capacity for osteogenic differentiation.

### Example 2: Evaluation of the Clinical Effect of a Single Injection of Cryopreserved Feline NSCs for the Care for Feline CGS Refractory to the Conventional Treatments and Having Undergone a Total Dental Avulsion or a Partial Dental Avulsion

### A. Programme of the Studies

This pilot clinical study is multicentric, non-randomised. The cats included in these studies (6 for total avulsion and 2 for partial avulsion) are owners' animals suffering from CGS, in a situation of therapeutic failure as specified in the inclusion criteria below. The inclusion criteria are specified in the following paragraph. There is no restriction in terms of sex, breed, weight of the animals.

Inclusion criteria: 1—Having inflammatory lesions specific to CGS (confirmed by a dentistry specialist); 2—Having tried other treatments such as ciclosporin, NSAIDs, antibiotics, IFN omega unsuccessfully; 3—Showing a persistence of the clinical signs after 2 months of treatment; 4—Dental extraction (partial or total) necessary and dating to more than 3 months; 5—Stopping the treatments with IFN or corticotherapy

Non-inclusion criteria: 1—Informed consent not signed by the owner; 2—Gestation, evolutive tumoral process, systemic infectious process, intercurrent disease that can interfere with the evaluation of the treatment; 3—Extreme physical weakening risking the life of the animal; 4—Administration of an unauthorised treatment during the study period (IFN or corticotherapy)

Criteria for exiting the study: 1—Restarting a treatment including ciclosporin, IFN, corticoids, lactoferrin during the study period; 2—Degradation of the general state during the trial leading to an extreme weakening risking the life of the animal; 3—Appearance of other intercurrent diseases that can interfere with the monitoring of the changes: evolutive tumoral process, systemic infectious process

The animals included in the study are evaluated by a veterinarian who rates the degree of severity of the diseases by using the Stomatitis Disease Activity Index (SDAI). This score allows to attribute a score of 0 (healthy animals) to 24 (severe CGS). Photographs of the mouth of the cat are taken at D0 and at the end of the study (6 months (M)) to verify the changes in the lesions. The owners of the animals included in the study receive a questionnaire to fill out to evaluate the activity, appetite, behaviour, and comfort of their animal. The animals receive the cell treatment the day of inclusion. The monitoring of the animals is planned at 15 days, 2 months, 3 months and 6 months post-inclusion. During the study, the animals are allowed to continue their non-steroidal anti-inflammatory treatment (NSAID) under the condition that the dose is specified in the study documents. The monitoring of the doses and the frequency of administration of the treatments during the study is an analysis criterion.

Table 1 summarises the calendar of the study and the various analyses carried out at each monitoring.

TABLE 1

| | | | perfusion | | | |
|---|---|---|---|---|---|---|
| | 1^{st} | | Post-treatment monitoring | | | |
| | visit | perfusion | D 15 | D 60 | D 90 | D 180 |
| Owner informed | X | | | | | |
| Informed consent | X | | | | | |
| obtained | | | | | | |
| Inclusion | X | | | | | |
| Perfusion of MSCs | | X | | | | |
| Monitoring | | X | | | | |
| Clinical evaluation | X | | X | X | X | X |
| by the veterinarian | | | | | | |
| investigator = SDAI | | | | | | |
| Weight | X | | X | X | X | X |
| Owner evaluation | X | | X | X | X | X |

The criteria for evaluating effectiveness are:

1. The evaluation of the clinical score (SDAI) during the study (15 d, 2 mo, 3 mo, 6 mo)
2. The evaluation of the owner score during the study (15 d, 2 mo, 3 mo, 6 mo)
3. The change in the medicinal needs of the cats during the study.

### B. Evaluation of the treatment

### Veterinarian Evaluation

The clinical evaluation is carried out by a veterinarian specialised in dentistry, who holds a specialist diploma from the European college (European Veterinary Dental College (EVDC)). The monitoring is programmed at 2 weeks, 2 months, 3 months and 6 months post-treatment. The veterinarian fills out a clinical monitoring form including the evaluation criteria described in detail below, the weight, the current treatments.

The main criterion for evaluating the effectiveness is the improvement of the clinical signs. This evaluation is based on the activity index of the CGS (SDAI) taking into account various parameters:
Oral inflammation (maxillary and mandibular) (0 none, 1 slight, 2 moderate, 3 severe);
Gingival inflammation (maxillary and mandibular) (0 none, 1 slight, 2 moderate, 3 severe);
The inflammation at the palatoglossal arch (0 none, 1 slight, 2 moderate, 3 severe);
The inflammation of the salivary gland (0 none, 1 slight, 2 moderate, 3 severe);
The oropharyngeal inflammation (0 none, 1 slight, 2 moderate, 3 severe);
The lingual or sublingual inflammation (0 none, 1 slight, 2 moderate, 3 severe).

### Owner Evaluation

The owners are also asked to carry out an evaluation of their cat on the criteria of activity, appetite, behaviour and comfort. Each parameter is scored from 0 to 3, resulting in a score of 12.

FIG. 4 shows the monitoring form filled out by the owners of the animals included in the study.

### Preparation of the Cellular Product

A dose of feline NSCs cryopreserved at −196° C. is thawed to 37° C. for 3-5 minutes in the laboratory the day before the administration of the treatment in conditions guaranteeing the asepsis of the product. An aliquot of the cellular product is sampled to verify the viability and the quantity of cells by the technique of trypan blue exclusion. The specifications of the product post-thawing are the following: viability greater than 80%, and total number of viable cells at least 10⁷. The precise number of viable cells counted is presented in table 2.

Table 2 below illustrates the results of the cell viability tests upon thawing.

TABLE 2

| | | |
|---|---|---|
| CAT ID | viability | number of viable cells |

total avulsion

| | | |
|---|---|---|
| 1 | 93.6 | 10.56 |
| 2 | 92.7 | 10 |
| 3 | 92 | 10.47 |
| 4 | 92 | 10.6 |
| 5 | 87.6 | 12.5 |
| 6 | 87.6 | 12.5 |
| average | 90.92 | 11.11 |
| standard | 2.64 | 1.10 |
| deviation | | |

partial avulsion

| | | |
|---|---|---|
| 1 | 91.1 | 10.59 |
| 2 | 92.3 | 13.23 |
| average | 91.70 | 11.91 |

The cells compliant with the specifications are transferred into a sterile sealable flask. The product packaged in its flask is transferred into a transport box with controlled temperature (4-12° C.). The treatment is delivered to the veterinary clinic for administration to the patient at D+1 or D+2.

### Administration of the Feline NSCs

The animal can be sedated for the control of the correct execution of the perfusion. The veterinarian chooses the most suitable site to fasten the veinous catheter to the animal. A bag of perfusion solution (Ringer's lactate) is used in combination with heparin. The bag is then homogenised by inversion, then the veterinarian connects the intravenous set to the bag of solution.

The cellular product (between 1.0·10⁷ and 1.4·10⁷ viable cells for an average of 1.2·10⁷.) is taken out of its package 15 min before the injection. The veterinarian homogenises the cellular suspension inside the flask by rotation. A syringe having a suitable volume with a needle of 18 G-20 G (70 mm) is prepared to suck up the cellular suspension inside the flask. The syringe is then connected onto the injection site at the bottom of the nozzle. The veterinarian slowly pushes the piston in order to eject approximately 0.2-0.5 ml of the suspension every 5 min. The totality of the feline NSCs is administered in approximately 20-30 min. The animals are awoken and kept at the clinic for 4 h minimum to verify their physiological parameters.

### Analysis of the Data

The main evaluation criterion is the SDAI clinical score defined by the veterinarian during each monitoring. The significance of the variability of the SDAI over time is compared to the score upon inclusion in the study (D0) using a non-parametric Friedman test.

### C. Results of the Study

Information on the subjects included in the study with complete or partial dental avulsion.

Eight cats were recruited over a period of 10 months in 3 investigating centres: 6 females and 2 males. The average age of the cats included in the study is 6.8 years (3-11 years). No cat was excluded from the study during the 6 months. Among the cats included in the study, 6 had undergone a total dental avulsion and 2 cats a partial dental avulsion. Four cats did not take any anti-inflammatories (NSAIDs) upon inclusion; 2 cats took NSAIDs on demand; and 2 cats followed a continuous treatment with NSAIDs.

Table 3 describes in detail the characteristics of the animals included in the study with complete or partial dental avulsion.

TABLE 3

| | | | | | |
|---|---|---|---|---|---|
| CAT | | age | | Investigating | Weight |
| ID | breed | (year) | gender | centre | (kg) |

Total avulsion

| | | | | | |
|---|---|---|---|---|---|
| 1 | Unkn. | 6 | female | 1 | 3.6 |
| 2 | European | 3 | female | 1 | 5.4 |
| 3 | Unkn. | 5 | female | 1 | 2.6 |
| 4 | European | 11 | female | 1 | 3 |
| 5 | European | 10 | female | 1 | 5.3 |
| 6 | Unkn. | 4.5 | female | 2 | 4.15 |

Partial avulsion

| | | | | | |
|---|---|---|---|---|---|
| 1 | European | 7 | male | 3 | 4.4 |
| 2 | European | 8 | male | 2 | 5.8 |

During the administration of the product, one cat vomited. An anti-vomiting drug was prescribed to it. No other undesirable event was observed during the perfusions and the monitoring of the animals included. The animals monitored during or after the perfusion did not show modifications of their physiological parameters: heart rate, respiratory rate.

Table 4 summarises the treatments administered by the owners of the cats during the 6 months of monitoring.

TABLE 4

| | | | | |
|---|---|---|---|---|
| inclusion | D 15 | D 60 | D 90 | D 180 |

TOTAL AVULSION

| | | | | | |
|---|---|---|---|---|---|
| 1 | none | none | none | none | none |
| 2 | NSAID | NSAID | NSAID | NSAID | NSAID |
| | Buprenor- | (1/day) | (1/day) | (¾ dose) | (½ dose) |
| | phine | | | | |
| 3 | NSAID | NSAID | none | NSAID | none |
| | | | | upon | |
| | | | | demand | |
| 4 | none | none | none | none | none |
| 5 | none | none | none | none | none |
| 6 | none | NSAID | NSAID | none | none |
| | | (1/month) | (1/month) | | |

PARTIAL AVULSION

| | | | | | |
|---|---|---|---|---|---|
| 1 | NSAID | unkn. | NSAID | unkn. | Diluted |
| | upon demand | | injection | | NSAID |
| | | | every 10 | | 1/month |
| | | | days | | |
| 2 | NSAID | NSAID | 3xmorphine | Antibiotics | IFN |
| | upon demand | upon | NSAID upon | for 1 week | |
| | gabapentin | demand | demand IFN | NSAID upon | |
| | | gabapentin | gabapentin | demand IFN | |
| | | | | gabapentin | |

Four of the six cats with total avulsion did not take NSAIDs upon inclusion. Three of these 4 cats did not take any treatment over the 6 months of monitoring (#1, 4 and 5). One cat resorted to 2 NSAID treatments over the first two months, then did not take any more (#6).

Two cats with partial avulsion and two cats with total avulsion took NSAID treatment upon inclusion. One of the cats with total avulsion markedly reduced the dose and the frequency of administration (#2), one cat with total avulsion did not take NSAIDs any more at 6 months (#3). The cats with partial avulsion either reduced the dose and the frequency of NSAIDs (#1) or stopped taking NSAIDs at 6 months (#2).

Change in the veterinarian clinical score of the cats included with complete dental avulsion.

Of the 6 cats having undergone a complete dental avulsion, 100% showed a favourable clinical change after the administration of feline NSCs. The clinical observations showed a reduction in the oral lesions as illustrated in FIG. 5.

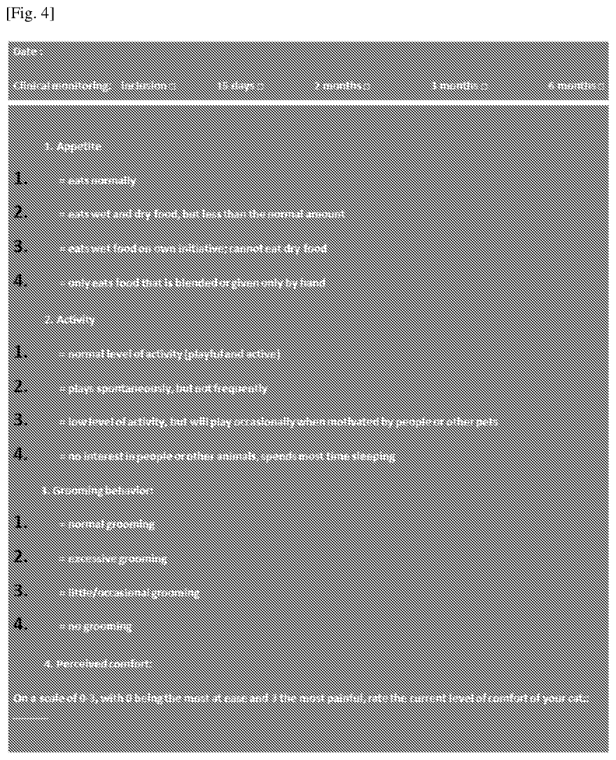

The reduction of the SDAI clinical score confirms the favourable change in the animals treated. FIG. 6 illustrates the change in the SDAI score over time. A non-significant reduction of the SDAI was observed 15 days post-treatment (median score at D15: 9.5 [4;14] vs median score at D0: 11 [9;13]). Two months post-treatment, a significant reduction in the score is observed (median score at M2: 5 [0;13] vs median score at D0: 11 [9;13]; p=0.0325). At three months post-treatment, a significant reduction in the score is observed (median score at M3: 6.5 [2; 9] vs median score at D0: 11 [9; 13]; p=0.0325). At 6 months, the signifcantness disappears (p=0.143) but the effect of the treatment seems to continue.

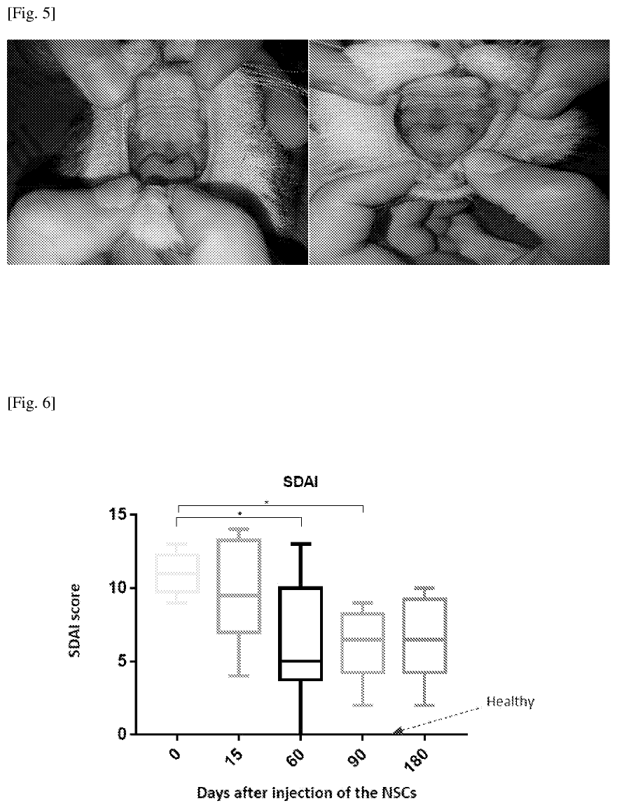

FIG. 7 illustrates the change in the SDAI per cat during the study. A complete resolution of the clinical signs 2 months post-administration in one cat (#4) is observed. In this cat, a slight oropharyngeal inflammation is observed which reappears at 3 and 6 months (score 2/24). At the monitoring at 3 months, a slight degradation in one cat (#3, score goes from 5 to 9) is observed, then the score decreases again to reach 7 at 6 months. An improvement in the state of the other cats is also observed.

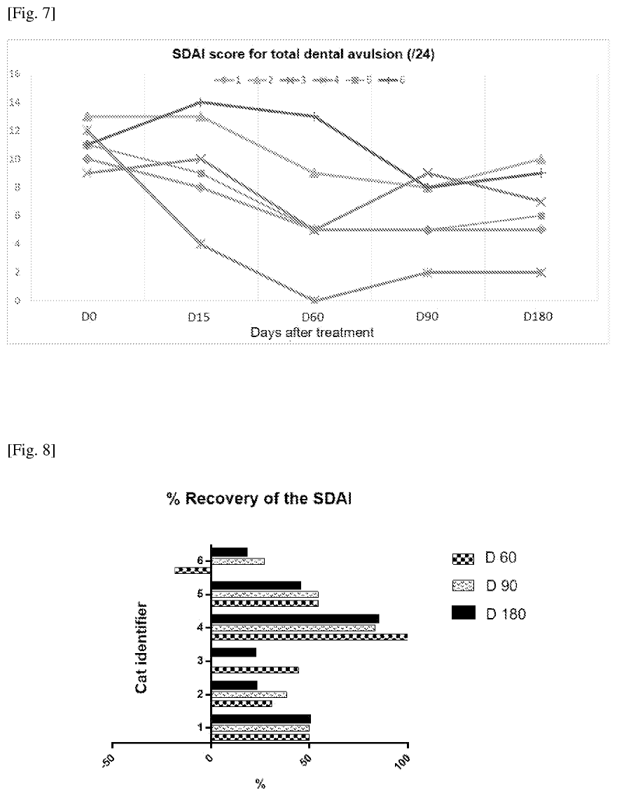

FIG. 8 represents the rate of clinical recovery of each animal having undergone a total avulsion at the various monitoring times. The recovery rate corresponds to the difference (in percentage) between the SDAI score evaluated at a monitoring time (2 months, 3 months, 6 months) and the SDAI score upon inclusion of the same animal, divided by the SDAI score upon inclusion. This ratio takes into account the improvement that occurred, if there is one. It is observed that a single cat (#6) slightly degraded at 2 months, then improved by 30% with respect to the inclusion starting at the monitoring at 3 months. One cat (#4) showed a complete recovery (clinical remission) at the monitoring at 2 months.

Owner monitoring of the cats included with complete dental avulsion.

The owner evaluation is based on 4 main criteria: appetite, activity, comfort and behaviour. Each parameter is graded out of /3, and thus allows to obtain a score of 12. The more the score decreases, the more the behaviour of the cat improves. The change is shown in FIG. 9. Between the inclusion and the monitoring at 6 months, an improvement of the score of all the cats included is observed except for cat #5, who only shows a transient improvement. Three cats are considered to be healthy starting at 15 days (#4), starting at 2 months (#1) or starting at 6 months (#6). One cat (#6) included with a score of 2 (few clinical signs) degraded at D15 and at 2 months to finally reach a healthy-cat score at 6 months (score=0). Finally, as observed with the SDAI scores, cat #3 degraded at 3 months to come back to its inclusion score then improved again between 3 and 6 months.

Change in the veterinarian clinical score of the cats included with partial dental avulsion.

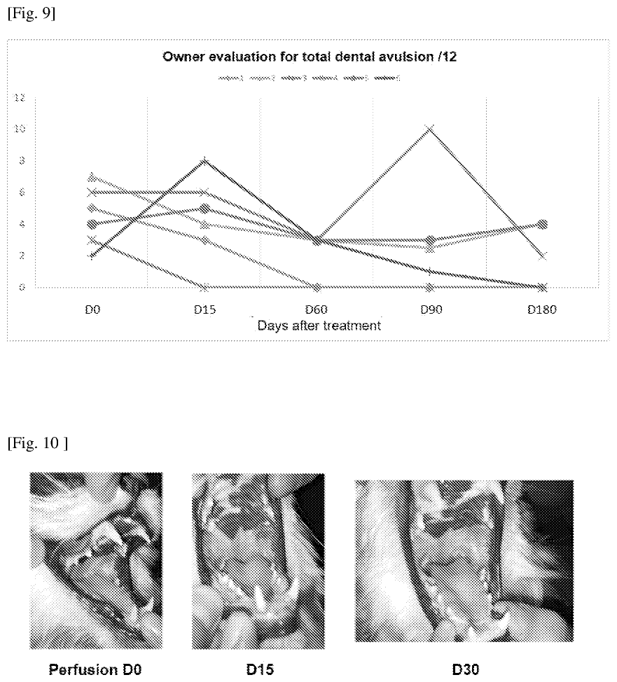

The 2 cats having undergone a partial dental avulsion also showed a favourable clinical change after the administration of feline NSCs. FIG. 10 shows a reduction in the oral lesions at 1 month after the injection.

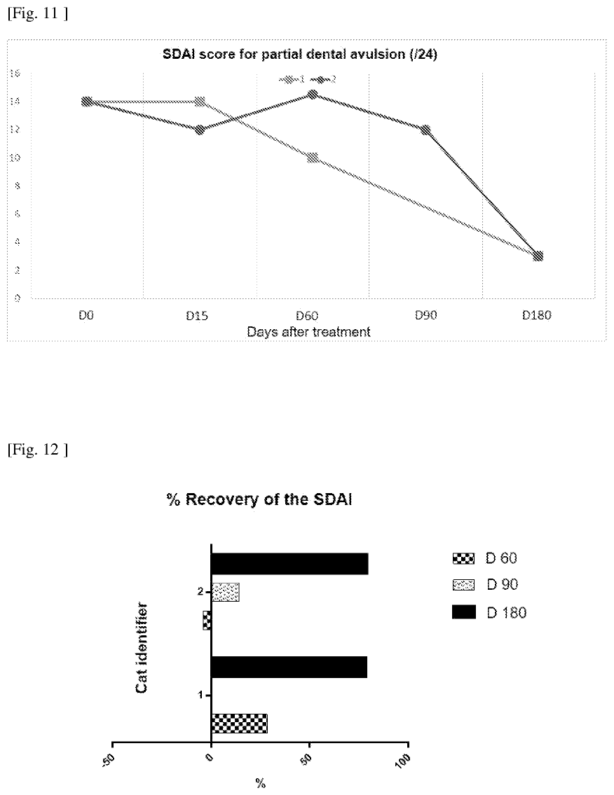

FIG. 11, showing the change in the SDAI for the two cats having undergone a partial dental avulsion shows an almost complete resolution of the clinical signs at 6 months post-administration with a score of 3. While cat #1 showed a fast clinical improvement starting at two weeks, cat #2 showed a recurrence of the symptoms at 2 months that greatly decreased afterwards.

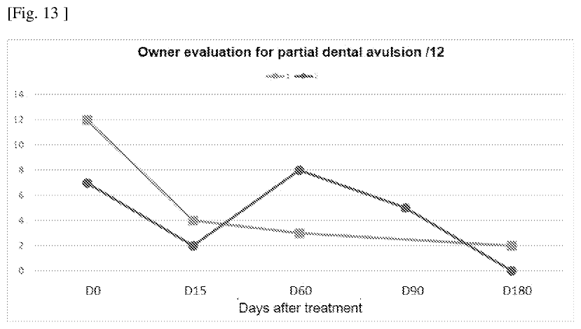

FIG. 12 shows the rate of clinical recovery of each animal having undergone a partial avulsion at the various monitoring times. For both cats, an improvement in the recovery rate can be observed starting at 90 days.

Owner monitoring of the cats included with partial dental avulsion.

FIG. 13 shows the change in the owner score for the two cats with partial dental avulsion. Here again, cat #2 showed a transient reduction in the symptoms to be later considered as healthy at 6 months after injection. The state of health of cat #1 quickly improved after two weeks to reach an owner score of 2 at 6 months.

### Conclusion

All of this data shows that a single perfusion of feline NSCs is safe for the animal and leads to a clinical improvement in all the cats diagnosed with chronic gingivostomatitis and not having responded to the various conventional treatments. Moreover, this clinical improvement can be observed starting at 2 months and the clinical benefit is observed over a period of at least 6 months.

### LIST OF THE CITED DOCUMENTS

### Patent Documents

For all intents and purposes, the following patent document(s) are cited:
US20160199414A1 (publication number); and
WO2017062475A1 (publication number).

### Non-Patent Literature

For all intents and purposes, the following non-patent element(s) are cited:
1. Verhaert, L., and Van Wetter, C. (2004). Survey of oral diseases in cats in Flanders. Vlaams Diergeneeskundig Tijdschrift 73,331-340.
2. Healey, K. A. E., Dawson, S., Burrow, R., Cripps, P., Gaskell, C. J., Hart, C. A., Pinchbeck, G. L., Radford, A. D., and Gaskell, R. M. (2007). Prevalence of feline chronic gingivo-stomatitis in first opinion veterinary practice. J. Feline Med. Surg. 9, 373-381.
3. Jennings, M. W., Lewis, J. R., Soltero-Rivera, M. M., Brown, D. C., and Reiter, A. M. (2015). Effect of tooth extraction on stomatitis in cats: 95 cases (2000-2013). J. Am. Vet. Med. Assoc. 246, 654-660.
4. Winer, J. N., Arzi, B., and Verstraete, F. J. M. (2016). Therapeutic Management of Feline Chronic Gingivostomatitis: A Systematic Review of the Literature. Front Vet Sci 3.
5. Uccelli, A., Moretta, L., and Pistoia, V. (2008). Mesenchymal stem cells in health and disease. Nat. Rev. Immunol. 8, 726-736.
6. Arzi, B., Mills-Ko, E., Verstraete, F. J. M., Kol, A., Walker, N. J., Badgley, M. R., Fazel, N., Murphy, W. J., Vapniarsky, N., and Borjesson, D. L. (2016). Therapeutic Efficacy of Fresh, Autologous Mesenchymal Stem Cells for Severe Refractory Gingivostomatitis in Cats: Autologous MSCs for Severe Refractory FCGS. Stem Cells Transl. Med. 5, 75-86.
7. Arzi, B., Clark, K. C., Sundaram, A., Spriet, M., Verstraete, F. J. M., Walker, N. J., Loscar, M. R., Fazel, N., Murphy, W. J., Vapniarsky, N., et al. (2017). Therapeutic Efficacy of Fresh, Allogeneic Mesenchymal Stem Cells for Severe Refractory Feline Chronic Gingivostomatitis. Stem Cells Transl. Med. 6, 1710-1722.
8. Bleiholder, A., Müle, M., Hechler, T., Bevins, S., vandeWoude, S., Denner, J., and Löchelt, M. (2011). Pattern of seroreactivity against feline foamy virus proteins in domestic cats from Germany. Vet. Immunol. Immunopathol. 143, 292-300.

9. Winkler, I. G., Löchelt, M., and Flower, R. L. (1999). Epidemiology of feline foamy virus and feline immunodeficiency virus infections in domestic and feral cats: a seroepidemiological study. J. Clin. Microbiol. 37, 2848-2851.
10. Arzi, B., Kol, A., Murphy, B., Walker, N. J., Wood, J. A., Clark, K., Verstraete, F. J. M., and Borjesson, D. L. (2015). Feline Foamy Virus Adversely Affects Feline Mesenchymal Stem Cell Culture and Expansion: Implications for Animal Model Development. Stem Cells Dev. 24, 814-823.
11. Allsopp, M. T. E. P., Lewis, B. D., and Penzhorn, B. L. (2007). Molecular evidence for transplacental transmission of Theileria equi from carrier mares to their apparently healthy foals. Vet. Parasitol. 148, 130-136.
12. Selich, A., Daudert, J., Hass, R., Philipp, F., von Kaisenberg, C., Paul, G., Cornils, K., Fehse, B., Rittinghausen, S., Schambach, A., et al. (2016). Massive Clonal Selection and Transiently Contributing Clones During Expansion of Mesenchymal Stem Cell Cultures Revealed by Lentiviral RGB-Barcode Technology. Stem Cells Transl. Med. 5, 591-601.
13. Lommer, M. J. (2013). Efficacy of cyclosporine for chronic, refractory stomatitis in cats: A randomized, placebo-controlled, double-blinded clinical study. J. Vet. Dent. 30, 8-17.

1. Pharmaceutical composition comprising a population of feline neonatal stromal cells (NSCs) and a pharmaceutically acceptable carrier.

2. Pharmaceutical composition according to claim 1, wherein said feline NSCs come from a sample of neonatal tissue.

3. Pharmaceutical composition according to claim 1, wherein said feline NSCs come from felines in the gestation period or from felines at the end of gestation.

4. Pharmaceutical composition according to claim 1, wherein said feline NSCs are placental feline NSCs.

5. Pharmaceutical composition according to claim 1, comprising between 5·10⁴ and 4·10⁷ cells/ml.

6. Pharmaceutical composition according to claim 1, characterised in that it is in frozen form.

7. A method of performing cell therapy in a subject in need thereof, comprising
administering to the subject a therapeutically effective amount of a population of NSCs or a pharmaceutical composition of claim 1.

8. The method of claim 7, wherein the cell therapy is an allogenic cell therapy.

9. The method of claim 7, wherein the cell therapy is a xenogeneic cell therapy.

10. A method of treating a feline stomatitis in a subject in need thereof, comprising
administering to the subject a therapeutically effective amount of a population of NSCs or a pharmaceutical composition of claim 1.

11. The method of claim 10, wherein the feline stomatitis is a feline chronic gingivostomatitis (CGS).

12. The method of claim 7, wherein a single dose of 3·10⁶ to 2·10⁷ feline NSCs, is administered.

13. The method of claim 10, wherein the treatment of the feline stomatitis is carried out in a feline not having undergone dental avulsion or having undergone a partial dental avulsion.

14. Ready-to-use injectable solution comprising a single dose of 5·10⁵ to 2·10⁷ feline NSCs in solution with a cryoprotectant.

15. The pharmaceutical composition of claim 1, wherein pharmaceutical composition comprises a cryoprotectant.

16. The pharmaceutical composition according to claim 2, wherein the neonatal tissue is from one or more of: one or more placentas; one or more umbilical cords; a sample of neonatal biological fluid.

17. The pharmaceutical composition according to claim 16, wherein the neonatal biological fluid is blood of one or more umbilical cords and/or amniotic fluid.

18. The pharmaceutical composition according to claim 5, wherein the pharmaceutical composition comprises i) between 5·10⁴ and 4·10⁷ cells/ml; or ii) 1·10⁷ cells/ml.

19. The method of claim 12, wherein the single dose is i) from 5·10⁶ to 1.5·10; or ii) 1·10⁷ feline NSCs.

20. The ready-to-use injectable solution of claim 14, wherein the single dose comprises 1·10⁷ feline NSCs.
